(19) United States
(12) Patent Application Publication
(10) Pub. No.: US 2022/0067839 A1
(43) Pub. Date: Mar. 3, 2022

(54) COLLECTING AND EVALUATING DRIVING DATA FOR EXTENDING AN INSURANCE OFFER

(71) Applicant: Rivian IP Holdings, LLC, Plymouth, MI (US)

(72) Inventors: Timothy Haugaard, Honolulu, HI (US); Thomas Rideout, Snohomish, WA (US); Michael P. Slattery, Irvine, CA (US); Joseph Frank Griffin, Bonita, CA (US)

(21) Appl. No.: 17/008,393

(22) Filed: Aug. 31, 2020

(51) Int. Cl. G06Q 40/08; G16H 40/67; G06K 19/07; G06K 7/10; G07C 5/02

(52) U.S. Cl. CPC G06Q 40/08; G16H 40/67; G07C 5/02; G06K 7/10366; G06K 19/0723

(57) ABSTRACT

Disclosed embodiments include systems and methods for recording and evaluating driving data for a particular operator and to extend an automobile insurance offer. In an illustrative embodiment, a system includes a driving data monitor configured to receive driving data on driving conduct for at least one operator of a vehicle. A driving data processing system is configured to receive the driving data from the driving data monitor and assign a driving score based on the driving data. The driving data processor determines if the driving score qualifies for a driving insurance offer from an insurance provider. The driving insurance offer is communicated via an interface in response to the score qualifying for the driving insurance offer.

### INTRODUCTION

The present disclosure relates to systems, and methods for recording and evaluating driving data for one or more operators and to extend an automobile insurance offer.

The statements in this section merely provide background information related to the present disclosure and may not constitute prior art.

Automobile insurance providers generally determine auto insurance rates for a driver based on the risk that the driver presents. Insurance providers commonly use a multivariate analysis to determine how likely a driver is to be involved in an accident based on compilations of historical data that may indicate how frequently drivers of the same age, gender, and similar determiners were involved in loss-related incidents and how costly those incidents proved to be. Insurance providers also may consider a driver's own historical driving record for loss-related incidents and whether he or she has been cited by police for speeding, reckless or distracted driving, or other moving violations. Rate quotations may vary significantly between different providers because the insurance providers' actuarial data may vary, as does the weight accorded to loss-related incidents on the driver's record.

Regardless of how insurance providers try to evaluate demographic data and a driver's individual record, the combined data may not provide a complete assessment of a particular driver's likelihood of becoming involved in a loss-related incident. To name a few examples, a driver may have reckless driving habits but, nonetheless, has managed to avoid loss-related incidents or being cited for moving violations. A young, inexperienced driver, may be deemed a high risk according to actuarial predictions and, thus, be considered costly to ensure, but that particular inexperienced driver may prove to be an extremely capable and careful driver. A formerly less-careful driver who had a past accident or moving vehicle infraction may have learned a lesson from such episodes and become a much more attentive and cautious driver.

It is of interest for drivers for their insurance rates to be based on an accurate assessment of their driving. It is also of interest for insurance providers to be able to accurately set rates for drivers based on their driving conduct to be able to provide appropriately priced rates based on the actual risk associated with the policies.

### SUMMARY

Disclosed embodiments include systems and methods for recording and evaluating driving data for a particular operator and to extend an automobile insurance offer to the operator.

In an illustrative embodiment, a system includes a driving data monitor configured to receive driving data on driving conduct for at least one operator of a vehicle. A driving data processing system is configured to receive the driving data from the driving data monitor and assign a driving score based on the driving data. The driving data processor determines if the driving score qualifies for a driving insurance offer from an insurance provider. The driving insurance offer is communicated via an interface in response to the score qualifying for the driving insurance offer.

In another illustrative embodiment, a vehicle includes a cabin configured to at least one occupant. A drive system is configured to motivate, accelerate, decelerate, stop, and steer the vehicle. A vehicle control system is configured to allow an operator to direct operations of the vehicle. A driving data monitor is configured to receive driving data on driving conduct. A driving analysis system is configured to receive the driving data from the driving data monitor, assign a driving score based on the driving data, determine if the driving score qualifies for a driving insurance offer from an insurance provider, and communicate the driving insurance offer via an interface responsive to the score qualifying for the driving insurance offer.

In a further illustrative embodiment, a computer-implemented method includes collecting driving data indicative of driving conduct to report data on at least one driving activity chosen from acceleration, deceleration, stopping, and steering of a vehicle. A driving score is assigned based on the driving data. It is determined if the driving score qualifies for a driving insurance offer from an insurance provider. The driving insurance offer is communicated in response to the driving score qualifying for the driving insurance offer.

Further features, advantages, and areas of applicability will become apparent from the description provided herein. It will be appreciated that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, with emphasis instead being placed upon illustrating the principles of the disclosed embodiments. In the drawings:

FIG. 1 is a schematic diagram of a system for recording and evaluating driving data and to extend an automobile insurance offer to the operator;

FIG. 2 is a schematic diagram of trip data collected by the driving data monitor of FIG. 1;

FIG. 3 is a schematic diagram of a record included in the trip data of FIGS. 1 and 2;

FIGS. 4A and 4B are schematic diagrams of the driving data analysis system of FIG. 1 using the trip data of FIGS. 1-3 to evaluate presentation of a driving insurance offer;

FIG. 5 is a block diagram of a vehicle supporting the system of FIG. 1 for possible presentation of the driving insurance offer;

FIG. 6 is a block diagram of a vehicle system communicating with a remote system to facilitate presentation of the driving insurance offer;

FIG. 7 is a block diagram of a computing system for performing functions of one or more on-board or remote systems;

FIG. 8 is a block diagram of an operator assist system that may be monitored or otherwise communicate with the system of FIG. 1;

FIG. 9 is a block diagram of sensor systems useable by the system of FIG. 1 to collect the trip data;

FIG. 10 is a perspective view of a cabin of a vehicle in communication with the system of FIG. 1; and

FIG. 11 is a flow chart of an illustrative method of recording and evaluating driving data to extend an automobile insurance offer to the operator.

### DETAILED DESCRIPTION

The following description is merely illustrative in nature and is not intended to limit the present disclosure, application, or uses. It will be noted that the first digit of three-digit reference numbers and the first two digits of four-digit reference numbers correspond to the first digit of one-digit figure numbers and the first two-digits of the figure numbers, respectively, in which the element first appears.

The following description explains, by way of illustration only and not of limitation, various embodiments of systems, apparatuses, and methods for evaluating driving behavior of an operator to determine if a driving insurance offer should be communicated to the operator based on the operator's driving behavior.

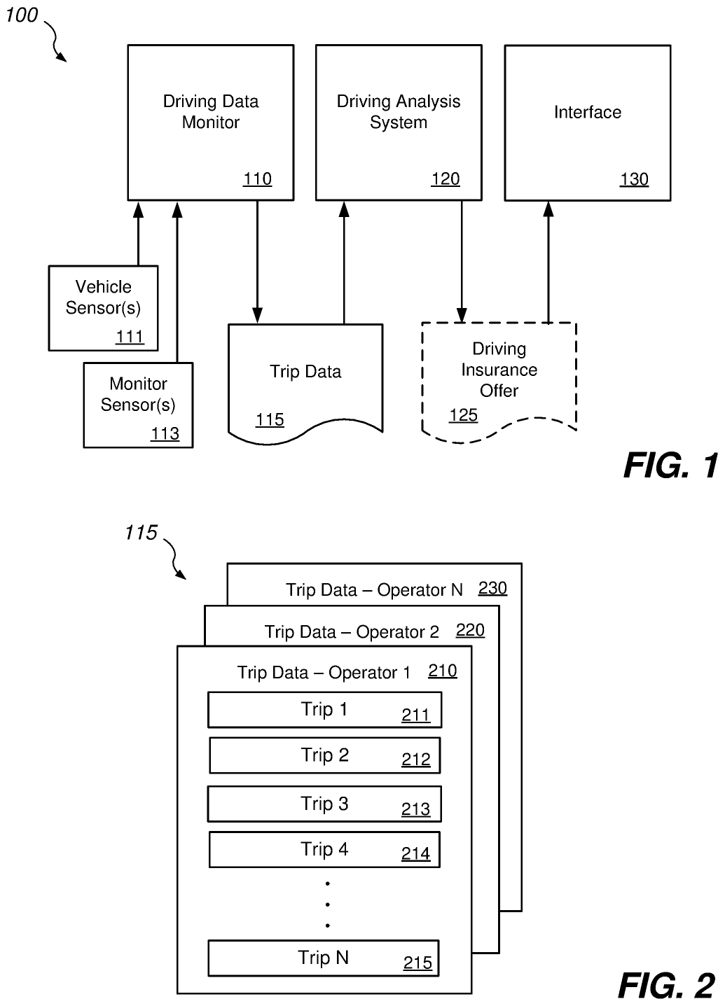

Referring to FIG. 1, various embodiments of the present disclosure include a system 100 for monitoring driving behavior of an operator and to potentially extend a driving insurance offer 125 to the operator. By way of overview, in various embodiments, the system 100 includes a driving data monitor 110, a driving analysis system 120, and an interface 130. As is further described below, the driving data monitor 110 is used to collect data about the behavior of a particular operator that is stored as trip data 115 that may be used to evaluate the operator's driving. The driving analysis system 120 evaluates data collected by the driving data monitor 110 to score the operator's driving and determine whether the operator may be deserving of a driving insurance offer 125 and the terms of that offer. (The driving insurance offer 125 is shown in dashed lines because the driving insurance offer 125 is generated only when the driving analysis system 120 determines it is appropriate to present the driving insurance offer 125.) The interface 130 may be used to communicate the driving insurance offer 125 to the operator and/or receive input from the operator related to the driving insurance offer 125.

Referring additionally to FIG. 1, in various embodiments, the driving data monitor 110 is configured to collect information about the driving behavior of the operator using one or more vehicle sensors 111 and/or one or more monitor sensors 113. The vehicle sensors 111 include sensors integrated into or mounted on the vehicle (not shown in FIG. 1). The vehicle sensors 111 may include sensors coupled to the drive system of the vehicle to measure velocity, acceleration, and braking. The vehicle sensors 111 also may include one or more cameras or other sensors configured to measure following distance to other vehicles, to determine whether the vehicle is staying within its lane, in what locations the operator drives and/parks, to what extent the operator employs operator assist features or automated driving features. The vehicle sensors 111 also may include other sensors to collect data regarding the driving behavior of the operator, including one or more inward-facing cameras or other devices to monitor for distracted driving, as described further below. The monitor sensors 113 may be housed within the driving data monitor 110 and, while they may be integrated into the vehicle, the monitor sensors 113 do not have to be coupled to the vehicle. The monitor sensors 113 may include accelerometers, global positioning system (GPS) devices, and other sensors that can monitor acceleration, cornering, braking, location, and other data without necessarily being coupled with or in communication with vehicle systems. The types of sensors included in the vehicle sensors 111 and monitor sensors 113 are described further below.

In various embodiments, the driving data monitor 110 may be an integral component of a vehicle, such as part of a control system (not shown in FIG. 1) that is engaged in operations of a vehicle. In various other embodiments, the driving data monitor 110 may include another computing system, such as a portable computer, tablet computer, smartphone, smartwatch, or other device that is transportable aboard the and/or in communication with sensors aboard the vehicle to monitor sensors that provide information about driving behavior. A movable driving data monitor 110 may communicate with the vehicle sensors 111 through wired communication provided via an interface within the vehicle or through wireless communications, such as Bluetooth, Wi-Fi, or other protocols.

By using a computing system that may be transported in or in communication with sensors aboard different vehicles, information about driving behavior for a particular operator may be collected even when the operator uses more than one vehicle. Being able to collect driving behavior information across multiple vehicles may be desirable when the operator is part of a household that owns multiple vehicles, when the operator uses car-sharing services, or when the operator travels regularly and the use of rental cars may constitute a considerable part of the operator's driving time.

The information collected by the driving data monitor 110 using the vehicle sensors 111 and monitor sensors 113 is collected in the trip data 115 that stores data about the operator's driving. The trip data 115 may be stored in the driving data monitor 110 until it is requested by and/or provided to the driving analysis system 120. The trip data 115 also may be stored in a separate data store aboard the vehicle or in storage on a network in communication with the driving data monitor 110 until requested by and/or provided to the driving analysis system 120. The trip data 115 also may be continually provided to and stored by the driving analysis system 120 for current or subsequent analysis.

The driving analysis system 120 assesses the trip data 115 to evaluate the operator to determine if the operator's driving behavior merits a driving insurance offer 125. In various embodiments, the driving analysis system 120 may be configured to assess any number of factors in determining whether the driving insurance offer 125 is presented to the operator. As referenced previously and explained in more detail below, the trip data 115 evaluated by the driving analysis system 120 presents driving behavior information indicative of the level of risk associated with insuring the driver. Just for example, the driving analysis system 120 reviews sensor data stored in the trip data 115 to determine how fast the operator drives, how hard the operator accelerates, brakes, and corners, how closely the operator follows other vehicles, and how well the operator stays within a chosen lane. The driving analysis system 120 also may analyze whether the operator travels on roads or parks in areas that present a higher risk of vehicle-related loss. The driving analysis system 120 also may evaluate the extent to which the operator relies on available operator assist features or automated driving that may help reduce loss-related incidents. The driving analysis system 120 also may assess other factors indicative of how likely the operator is to be involved in a loss-related incident, such as distracted driving.

In various embodiments, and as described further below, a driving score may be assigned to each trip taken by the operator and/or to a series of trips taken by the operator. The driving score is predicated on consideration of factors indicative of the operator becoming involved in a loss-related incident, such as those previously described. For example, the driving score may include factors such as an extent to which the operator has employed available operator assist capabilities or has used automated driving, which may reduce the risk of an incident. In addition, the driving behavior of the operator may be evaluated by comparing driving speed to a posted speed limit or a speed limit that is adjusted based on detected road or weather conditions. The driving behavior may be evaluated based on rapid acceleration that may be excessive based on a predetermined acceleration limit. The driving behavior also may be evaluated based on hard braking that decelerates the vehicle more quickly than a predetermined deceleration limit. Detection of distracted driving also may be used in evaluating driving behavior. Based on an assessment of such factors, a driving score may be generated that is indicative of the likelihood of the operator becoming involved in a loss-related incident.

When the driving score based on the trip data 115 for a specified number of trips indicates that the operator presents a risk of loss-related incident within a range acceptable to an insurance provider, the driving analysis system 120 may generate the driving insurance offer 125. The driving insurance offer 125 is then presented via the interface 130 to the operator. The interface 130 may be part of the driving data monitor 110, the driving data processing system 120, or another computing system that is integrated with the vehicle, transportable aboard the vehicle, or in communication with the vehicle. The interface 130 also may be a completely separate computing device that the operator may use, such as a computer, smartphone, or other device where the operator receives emails or other electronic communications.

In various embodiments, the driving insurance offer 125 may be generated for presentation to the driver in various different situations. For example, the operator may request a driving insurance offer 125 from an insurance provider associated with the system 100 and, if the driving analysis system 120 determines that the operator is worthy of an offer, the system 100 will generate the driving insurance offer 125. On the other hand, the driving insurance offer 125 may be generated independent of an operator request. For example, although the operator may have declined a previous offer from the insurance provider, if the driving analysis system 120 determines from the trip data 115 that the operator's driving behavior is better than was previously estimated or has improved over time, an additional or updated driving insurance offer 125 may be presented to the operator at a potentially more desirable rate. In any case, the driving insurance offer 125 presented is based at least in part on actual, individualized information about the operator's driving behavior collected by the driving data monitor 110 and analyzed by the driving analysis system 120, rather than merely being projected from actuarial tables or previous incident and police records.

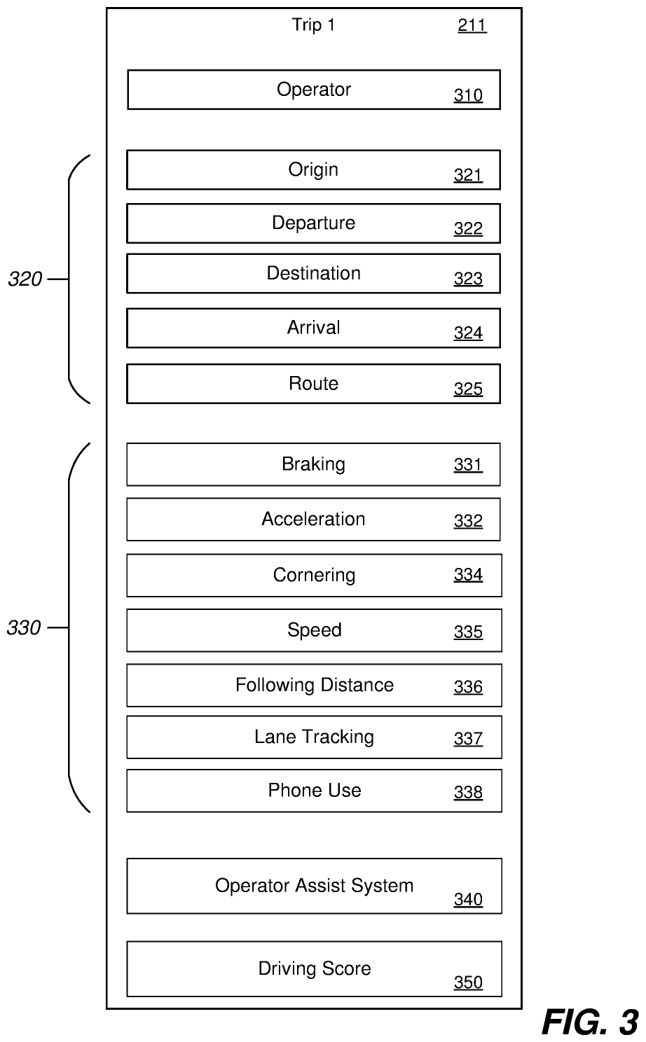

Referring to FIG. 2, the trip data 115 may include records 211-215 for each trip including Trip 1 211, Trip 2 212, Trip 3 213, Trip 4 214, etc., through Trip N 215 that is monitored by the driving data monitor 110. Because the driving data monitor 110 may be integral to a vehicle (not shown in FIG. 2) or is otherwise aboard a vehicle that may be driven or controlled by different operators, the trip data 115 may include driving data sets 210, 220, and 230 for each of multiple different operators in a household or who otherwise are monitored by the driving data monitor. Thus, for example, the driving data sets may include a driving data set for Operator 1 210, a driving data set for Operator 2 220, etc., through a driving data set for Operator N 230. The driving data sets 210, 220, and 230 may literally be maintained separately as shown in FIG. 2 or the driving data sets 210, 220, and 230 may be maintained in linked lists or in portions of a data storage array from which they may be logically accessed as shown in FIG. 2. It will be appreciated that maintaining separate data driving sets for separate drivers may is useful. Driving insurance is traditionally contracted for individuals based on a specific vehicle, their demographic information and general driving history; this is even more pertinent in various embodiments that monitor detailed driving behavior for individuals so as to be able to specifically assess the risk of loss associated with the driving behavior of individual operators as described herein. As further described below, in various embodiments, the driving data monitor 110 is configured to identify the operator of the vehicle to help ensure that the driving data is correctly associated with the appropriate operator.

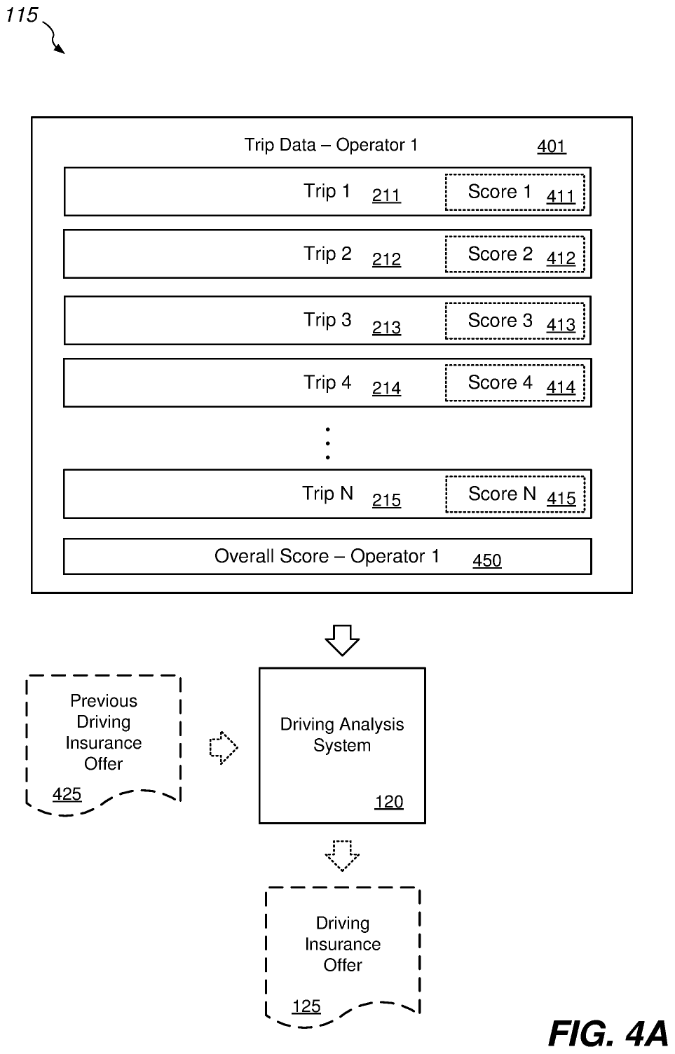

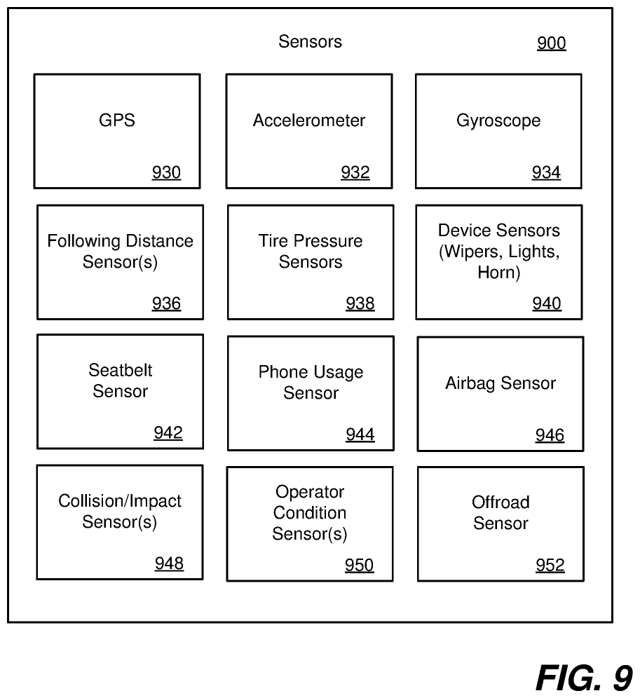

Referring to FIG. 3, a record of an individual trip, such as Trip 1 211 may store a wide range of information collected by the driving data monitor 110 from the vehicle sensors 111 and/or the monitor sensors 113. The record 211 may include an operator identifier 310 to identify the operator for the trip, although such an operator identifier 310 may be omitted if, as shown in FIG. 2, records for different operators are collected in different data sets such driving data sets 210, 220, 230. The record 211 may include different categories of information, such as location and time data 320, travel data 330, and data from an operator assist system 340. The operator assist system 340, which may provide assistance up to and including automated driving, includes automatic emergency braking, lane keeping assist, and other features described in detail below with reference to FIG. 9. The data from the operator assist system 340 includes information as to when one or more features of the operator assist system 340 were engaged by the operator and the extent to which the features of the operator assist system 340 initiated actions to assist the operator to avoid potential hazards, such as collisions, unintended lane departures, etc. The record 211 may include a field to store a driving score 350 assigned to the record 211 representing an overall evaluation of the trip represented therein.

The location and time data 320 may provide information about where and when the vehicle was operated. It will be appreciated that the types of roads travelled, considering the roads' type, congestion, and condition, and driving in various weather conditions, may factor into an assessment of driving behavior. The locations where the vehicle has been parked, and the times of travel all may be relevant in considering risk of loss that may influence a driving insurance offer 125. For example, whether an operator travels a long distance over a congested, divided highway at rush hour in a major city may represent a different risk situation than someone who travels a short distance across surface streets in a small community during evening or nighttime hours when roads are less congested. Roads with bad surface conditions, for example, resulting from lack of maintenance or ongoing freeze-thaw conditions in colder climates may pose a greater risk of collision-free damage to a vehicle than well-maintained streets or roads located in warmer climates. Parking in a neighborhood with a higher vehicle-related crime rate—either as a point of origin or a destination—is relevant to determining a risk associated with the trip. Thus, location and time data 320 may be considered in evaluating risk of loss that may factor into a driving insurance offer 125.

Referring additionally to FIG. 3, the location and time data 320 may include an origin identifier 321 that indicates the starting point of the trip as well as a destination identifier 323 that specifies the destination of the trip. Again, the conditions attending where the vehicle is parked may having a bearing on evaluating risk of loss. Similarly, the time of departure 322 and time of arrival 324 not only may be indicative of congestion and other traffic conditions but also may be representative of the length of the trip, all of which may contribute to a risk and possible severity of loss associable with the trip. In addition, route data 325 indicating whether the operator chose to take surface streets, local highways, divided highways, or other routes may be indicative of the risk of loss that may be expected.

Further referring to FIG. 3, the travel data 330 may be further illustrative of the risk of loss associated with a particular operator. Various sensors as previously referenced and further described below may be used to collect data on how well the operator controls the vehicle in addition to the time and location where the operator operates the vehicle. Accelerometers and/or gyroscopes may be used to collect data on braking 331, acceleration 332, and cornering 334. GPS sensors also may be used to monitor these data elements. Similarly, sensors integrated into the vehicle also may measure speedometer changes to collect data on braking 331 and acceleration 332. Data may be collected from the speedometer or from GPS sensors to determine at what speed 335 the operator travels and whether that is in accordance with posted limits (which may be stored in mapping data) and other conditions including, for example, traffic and weather conditions.

Additional vehicle sensors 111 or monitor sensors 113 may be used to collect additional information, such as following distance 336 between the vehicle and other vehicles on the road, lane tracking 337 indicative of how much the operator strays across edges of lanes or edges of roadways, phone usage 338 which may be indicative of whether the operator is distracted from controlling the vehicle, and other information gathered by additional sensors, as described further below.

In various embodiments, the extent to which features of the operator assist system 340 are used may be considerable in evaluating risk of loss associable with a trip. For example, engaging automatic braking for collision avoidance, lane departure warnings or lane-keeping assist systems, blindspot detection systems, and other systems that may prevent incidents may help reduce the risk of a loss-related incident while the operator is controlling a vehicle. Similarly, the use of automated driving, which may be safer than manual operation of a vehicle; use of automated driving, also may reduce the risk of a loss-related incident. Thus, the use of operator assist features and/or automated driving may be logged in the use of operator assist system field 340 for assessment of the operator by the driving analysis system 120 (FIG. 1).

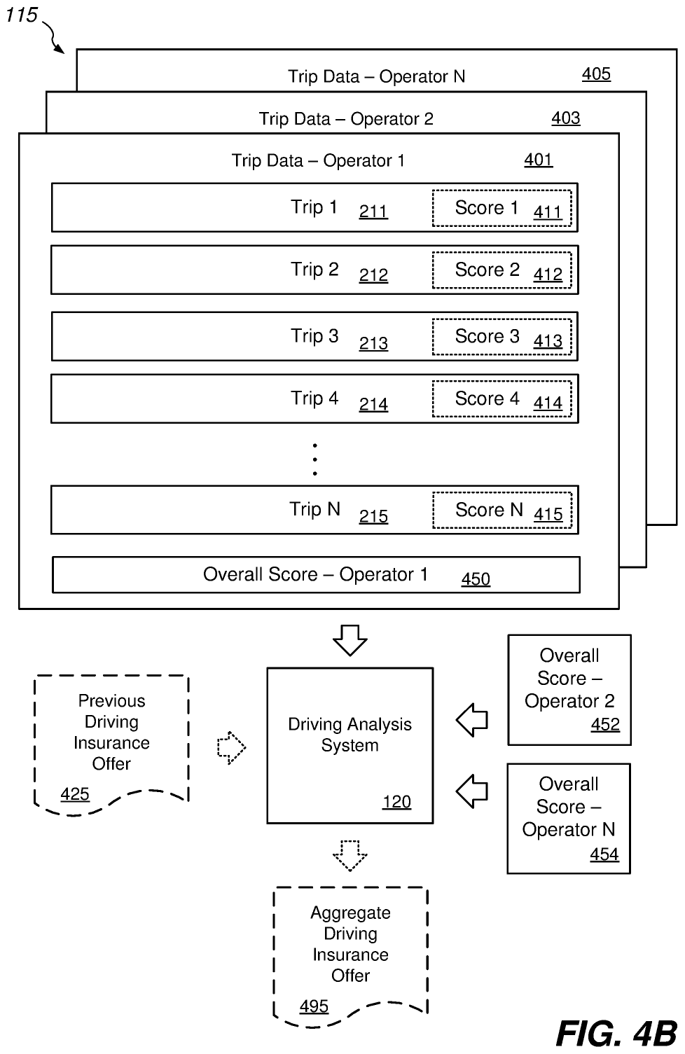

Based on the information collected in the record for trip 1 211, in various embodiments, a driving score 350 may be assigned by the driving analysis system 120 (FIG. 1) to the trip. On the other hand, as described below with reference to FIG. 4, driving scores may be tabulated and assigned to a series of trips rather than being assigned to individual trips. In either case, driving score 350 is indicative of the risk or a relative risk of the operator incurring loss-related incidents. The driving score 350 thus, may be used in determining whether to offer a driving insurance offer 125 and what the terms of the driving insurance offer 125 might be.

Referring to FIG. 4A, the driving analysis system 120 scores or otherwise evaluates the trip data 115 from a number of trips to determine whether to generate a driving insurance offer 125 and what the terms of the driving insurance offer may be. The trip data 115, as described with reference to FIG. 1 may include separate stores of trip data for each of a number of operators. Alternatively, the trip data 115 may include a collective store of data for all the operators monitored by the driving data monitor 110 but are associated with individual operators by an operator identifier 310 (FIG. 3) to allow the driving analysis system 120 to later access the information for a particular operator. In the example of FIG. 4, the driving analysis system 120 accesses a store of trip data for operator 1 401 which includes records of Trips 1-N 211-215, as described with reference to FIG. 1.

As described with reference to FIG. 3, in various embodiments, individual scores 1-N 411-415 may have been assigned for each of Trips 1-N 211-215 as previously described with reference to FIG. 3. (The individual scores 1-N 411-415 are represented by dotted lines to indicate the individual scores 1-N 411-415 are an optional feature). The scores 1-N 411-415 may be averaged or otherwise statistically evaluated to assign an overall score 450 for the operator for the collection of data from Trips 1-N 211-215. In various embodiments, the trip data from Trips 1-N 211-215 may be evaluated collectively in order to assign the overall score 450. As previously stated, the total driving score 450 is indicative of the risk or a relative risk of the operator incurring loss-related incidents.

Based on the overall driving score 450, the driving analysis system 120 determines whether to generate and present the driving insurance offer 125 to the operator. As previously described, whether the driving insurance offer 125 is generated and presented may be triggered by an operator request for the driving insurance offer 125. In various embodiments, periodically, the driving analysis system may generate and present the driving insurance offer 125. In other embodiments, when the aggregate score 450 indicates that the driving insurance offer 125 may be at a lower cost or otherwise be appealing to the operator in comparison to a previous driving insurance offer 425 presented to the operator, the driving insurance offer 125 may be generated and presented to the operator. In other words, the driving analysis system 120 may repeatedly or continually review the trip data 115 for the operator and generate the driving data insurance offer 125 when the driving analysis system 120 determines that it can make a better offer to the operator than the previous driving insurance offer 425.

In various embodiments, the previous driving insurance offer 425 may have been an offer based on a generalized multivariate assessment based on the operator's descriptive factors and/or driving history. In various embodiments, the previous driving insurance offer 425 also may have been made on detailed analysis performed by the driving analysis system 120, but after the passage of time, the driving analysis system 120 may generate a more favorable analysis of the operator that would support generation of the driving insurance offer 125.

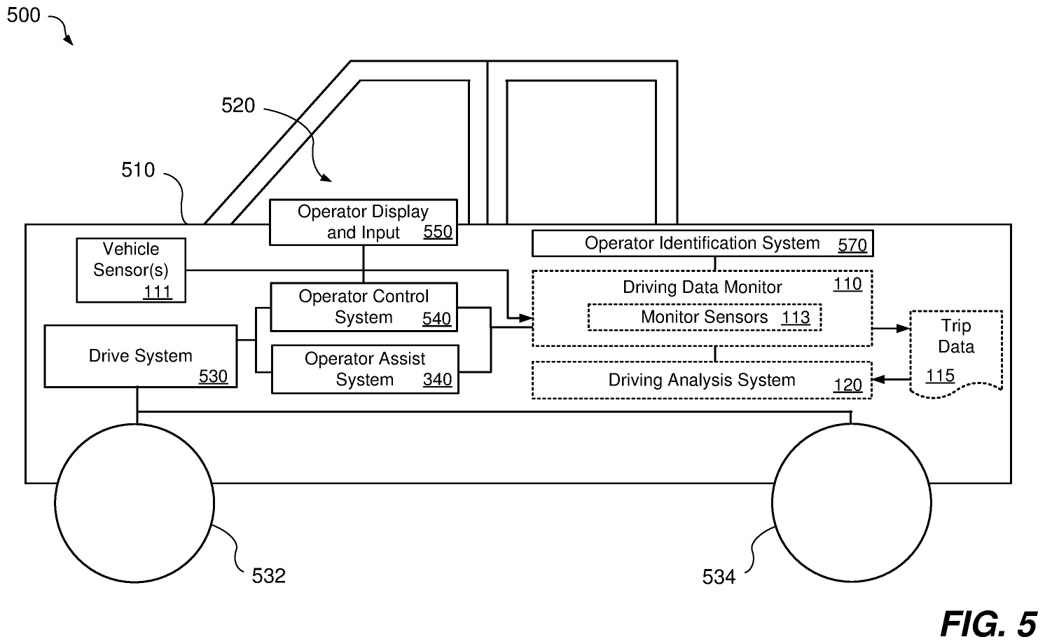

In addition, using the data gathered by the driving data monitor 110 and analyzed by the driving analysis system 120, one or more suggestions for safer driving may be presented to the operator. Using an operator display and input system as described below with reference to FIG. 5 or another interface for communicating with the operator, the driving analysis system 120 may provide suggestions. The suggestions may include using operator assist features or an automated driving system, as described further below. Specific suggestions about traveling a route or parking at a location that is less likely to result in a loss-related incident may be presented. Specific suggestions about speed, following distance, and other driving behaviors may be presented to the operator. Such suggestions may help the operator to avoid a loss-related incident which, in turn, may result in a more favorable driving insurance offer 125 being available to the operator.

Referring to FIG. 4B, the driving analysis system 120 also may score or otherwise evaluate the trip data 115 from a number of trips for multiple operators that share one or more vehicles to determine whether to generate a driving insurance offer 125 and what the terms of the driving insurance offer may be. As previously described, the trip data 115, may include separate stores of trip data for each of a number of operators, including stores of trip data for operator 1 401, operator 2 403, through operator N 405. As also previously described, the trip data 115 may include a collective store of data for all the operators monitored by the driving data monitor 110 but are associated with individual operators by an operator identifier 310 (FIG. 3) to allow the driving analysis system 120 to later access the information for each of the operators. In the example of FIG. 4B, the driving analysis system 120 accesses stores of trip data for each of operator 1 401, operator 2 403, through operator N 405.

In various embodiments, the stores of trip data for each of operator 1 401, operator 2 403, through operator N 405 may each be individually evaluated and/or scored to generate individual overall scores 450, 452, and 454 for each of the operators, just as the overall driving score 450 was determined for operator 1 as previously described with reference to FIG. 4A. The individual overall scores 450, 452, and 454 then may be combined to yield an aggregate score 455 for all of the operators that share the vehicle. The aggregate score 455 then may be used by the driving analysis system 120 to determine whether an aggregate driving insurance offer 495 should be made. In various embodiments, it will be appreciated that, instead of generating individual overall scores for each of the operators, each of the trip scores may be combined as a whole to generate the aggregate score 495. It will be appreciated that a determination of the aggregate driving insurance offer 495 may be made based on a combination of overall driving scores 450, 452, and 454 for individual operators, as described with reference to FIG. 4A, and the aggregate driving score 455.

Referring to FIG. 5, various embodiments include systems that may be integral to, carried aboard, or otherwise in communication with a vehicle 500. The vehicle 500 includes onboard systems to operate the vehicle 500, to help monitor the driving behavior of the operator, and perform other functions. The vehicle 500 may include a car, truck, sport utility vehicle (SUV), or similar vehicle for on-road and/or off-road travel. The vehicle 500 includes a body 510 that supports a cabin 520 to accommodate an operator and, optionally, one or more passengers, as well as the previously-referenced systems

The vehicle 500 includes a drive system 530 that, in concert with front wheels 532 and rear wheels 534, motivates, accelerates, decelerates, stops, and steers the vehicle 500. In various embodiments, the drive system 530 is directed by an operator control system 540 and/or an operator assist system 340. The operator control system 540 works in concert with an operator display and input system 550 within the cabin 520. The operator display and input system 550 includes all the operator inputs, including the steering controls, the accelerator and brake controls, and all other operator input controls. The operator display and input system 550 also includes the data devices that provide information to the operator, including the speedometer, tachometer, fuel gauge, temperature gauge, and other output devices. When the vehicle 500 is equipped with the operator assist system 340, the operator display and input system 550 also allow the operator to control and interact with the operator assist system 340.

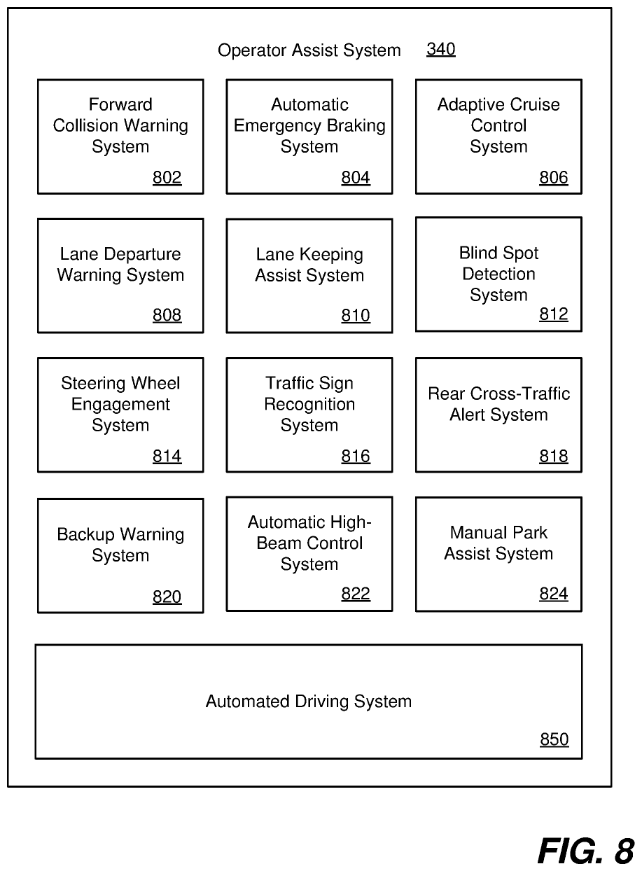

The operator assist system 340 includes available automated, self-driving capabilities or other features that assist the operator, such as a forward collision warning system, an automatic emergency braking system, a lane departure warning system, and other features described below with reference to FIG. 8. The operator assist system 340 thus partially or fully controls operation of the vehicle 500 and/or provides warnings to the operator that may help the operator to avoid accidents. The operator assist system 340 thus, instead of or in concert with the operator control system 540, engages the drive system 530 to motivate, accelerate, decelerate, stop, and steer the vehicle 500 and/or to provides information and warnings to the operator of the vehicle 500.

In various embodiments, the vehicle 500 also may include the driving data monitor 110, the trip data 115, and the driving analysis system 120. Each of the driving data monitor 110, the trip data 115, and the driving analysis system 120 are represented by dotted lines to indicate that these features may be integrated into or part of the vehicle 500 or may be supported by one or more separate systems that are transportable aboard the vehicle 500 or may be supported by remote systems in communication with the vehicle 500.

The driving data monitor 110, as previously described, may communicate with one or more vehicle sensors 111 that are mounted on or within the vehicle 500. The vehicle sensors 111 may include sensors coupled to the drive system 530 of the vehicle to measure velocity, acceleration, and braking. The vehicle sensors 111 also may include one or more cameras or other sensors configured to measure following distance to other vehicles, to determine whether the vehicle is staying within its lane, in what locations the operator drives and parks, to what extent the operator employs operator assist features or automated driving features, and other information that may represent the driving behavior of the operator. The one or more cameras or other sensors also may be used to evaluate and monitor weather and road conditions, for example, to determine whether the road is dry, wet, or icy. The driving data monitor 110 also may employ its own monitor sensors 113 (FIG. 1) that may be housed within the driving data monitor 110, including accelerometers, global positioning system (GPS) devices, and other sensors, as previously described. The types of sensors included in the vehicle sensors 111 and monitor sensors 113 are described further below.

The trip data 115 collected by the driving data monitor 110 may be stored in the driving data monitor 110, stored in the driving analysis system 120, or stored in a separate data store aboard the vehicle 500 or maintained on a network in communication with the vehicle 500, as further described below.

The driving analysis system 120, as previously described with reference to FIGS. 1-4, also is represented in dotted lines because the driving analysis system 120 may be integrated into or part of the vehicle 500 or may be supported by one or more separate systems that are transportable aboard the vehicle 500 or by one or more remote systems in communication with the vehicle 500 over a network, as further described below. When the driving analysis system 120 generates the driving insurance offer 125 (FIGS. 1 and 4; not shown in FIG. 5), it may be presented via an interface including the operator display and input system 550 aboard the vehicle, or via a separate interface 130 as previously described with reference to FIG. 1.

Because the tracking of the trip data 115 by the driving data monitor 110 is associated with a particular operator, it is desirable to identify the operator of the vehicle 500 to ensure that the trip data 115 is associated with the proper operator. To this end, in various embodiments, the vehicle 500 also includes an operator identification system 570 that may communicate with the driving data monitor 110 to provide operator identification information to the driving data monitor 110. As further described below, various embodiments of the operator identification system 570 may identify the operator based on the driver's seat being moved to a position used by a particular operator, by detecting a key fob assigned to a particular operator, by identifying a presence in the cabin 520 of a cell phone owned by a particular operator, by using an imaging system to identify the operator, or through other devices and methods.

In addition to the onboard systems, various embodiments may communicate with one or more remote computing systems to perform the functions herein described. For example, it may be desirable to communicate the trip data 115 to a remote computing system or store the trip data on a remote computing system. The trip data 115 may be made available to the driving analysis system 120 which also may be supported on a remote computing system.

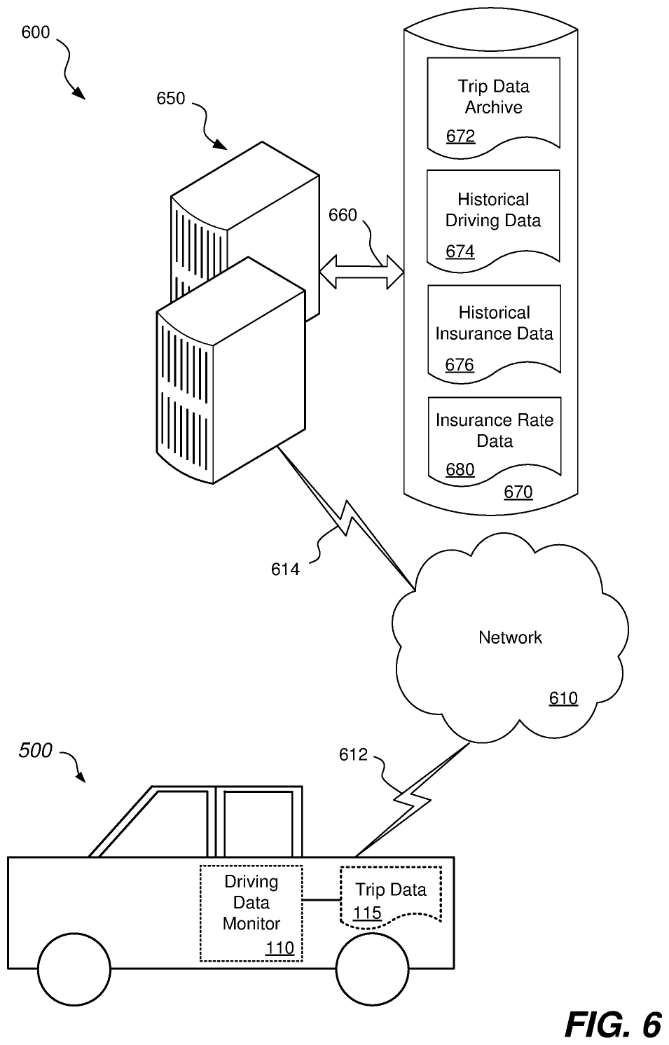

Referring to FIG. 6, an operating environment 600 of the vehicle 500 may include a remote computing system 650 that is configured to communicate with systems aboard the vehicle 500, such as the driving data monitor 110. For example, the driving data monitor 110 may send the trip data 115 to the remote computing system 650 and receive data, such as a driving insurance offer 125 (FIGS. 1 and 4), from the remote computing system 650 via a network 610. The driving data monitor 110 and/or other systems aboard the vehicle 500 may connect to the network 610 via a wireless communications link 612, such as a satellite, cellular, or Wi-Fi communications link. When the vehicle 500 is parked, it is also possible that the vehicle 500 may be coupled to the network 600 a wired network link, such as a universal serial bus (USB) or Ethernet connection, which may be part of a wiring harness used to charge the vehicle 500 when it is an electric or hybrid vehicle. The remote computing system 650, which may include a server or server farm, also communicates with the network 610 over wired and/or wireless communications links 614. The remote computing system 650 may access programming and data used to perform its functions over a high-speed bus 660 with data storage 670.

Information maintained in the data storage 670 includes a trip data archive 672 which may be used to store trip data from a number of trips, as described with reference to FIG. 4. The data storage 670 may also include historical driving data 674, including personal data or driving history data about operators that may be used together with driving behavior data represented in the trip data to generate a driving insurance offer. The data storage 670 also may include historical insurance data 676 about operators. For example, as previously described, before presenting a driving insurance offer 125, it may be desirable to see if a proposed driving insurance offer 125 represents a lower cost or otherwise more advantageous deal for an operator. The data storage 670 also may include insurance rate data 680 as may be needed to determine what rates, charges, deductibles, or other parameters to include in a driving insurance offer 125.

Using the many different types of information maintained in the data storage 670, the remote computing system 650 also may be configured with programming instructions to support the functions of the driving analysis system 120 (FIGS. 1 and 4). Thus, in various embodiments, the driving data monitor 110 may be integrated with or carried aboard the vehicle 500 to gather the trip data 115 and communicate the trip data 115 via the network 610 to the remote computing system 650 that performs the functions of the driving analysis system 120. If the driving analysis system 120 determines that the driving insurance offer 125 (FIGS. 1 and 4), the driving insurance offer 125 may be communicated via the network 610 to the vehicle or to another interface (FIG. 1) to present the driving insurance offer 125 to the operator. In other various embodiments, the driving data monitor 110, the trip data 115, and the driving analysis system 120 may be integrated within or carried aboard the vehicle 500, and the driving analysis system 120 may access the remote computing system 650 to retrieve the historical insurance data 676, the insurance rate data 680, or other information that the driving analysis system 120 may use to generate the driving insurance offer 125. The driving data monitor 110, the driving analysis system 120, and their functions to collect and evaluate the trip data 115 and generate the driving insurance offer 125 are not limited to any particular configuration or location of the components or the data that each uses or generates.

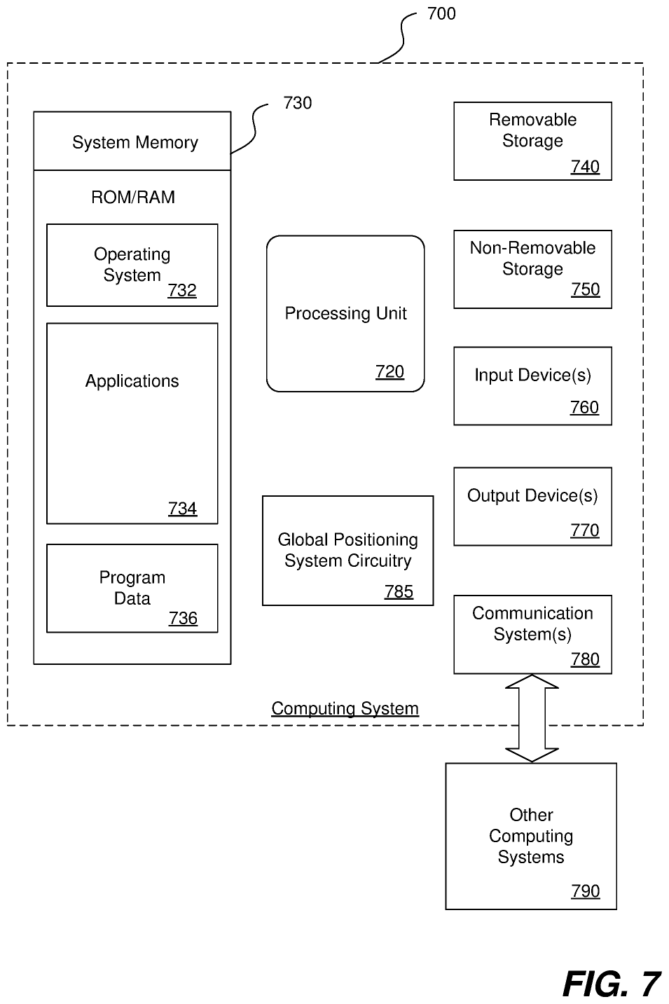

Referring to FIG. 7, and given by way of example only and not of limitation, some form of a computing system 700 may be used for the computing systems aboard the vehicle 500 (FIG. 5) or at the remote computing system 650 (FIG. 6). In various embodiments, the computing system 700 typically includes at least one processing unit 720 and a system memory 730. Depending on the exact configuration and type of computing device, the system memory 730 may be volatile memory, such as random-access memory (“RAM”), non-volatile memory, such as read-only memory (“ROM”), flash memory, and the like, or some combination of volatile memory and non-volatile memory. The system memory 730 typically maintains an operating system 732, one or more applications 734, and program data 736. The operating system 732 may include any number of operating systems executable on desktop or portable devices including, but not limited to, Linux, Microsoft Windows®, Apple OS®, or Android®, or a proprietary operating system.

The computing system 700 may also have additional features or functionality. For example, the computing system 700 may also include additional data storage devices (removable and/or non-removable) such as, for example, magnetic disks, optical disks, tape, or flash memory. Such additional storage is illustrated in FIG. 7 by removable storage 740 and non-removable storage 750. Computer storage media may include volatile and non-volatile, removable and non-removable media implemented in any method or technology for storage of information, such as computer-readable instructions, data structures, program modules or other data. The system memory 730, the removable storage 740, and the non-removable storage 750 are all examples of computer storage media. Available types of computer storage media include, but are not limited to, RAM, ROM, EEPROM, flash memory (in both removable and non-removable forms) or other memory technology, CD-ROM, digital versatile disks (DVD) or other optical storage, magnetic cassettes, magnetic disk storage or other magnetic storage devices, or any other medium which can be used to store the desired information and which can be accessed by the computing system 700. Any such computer storage media may be part of the computing system 700.

The computing system 700 may also have input device(s) 760 such as a keyboard, mouse, stylus, voice input device, touchscreen input device, etc. Output device(s) 770 such as a display, speakers, printer, short-range transceivers such as a Bluetooth transceiver, etc., may also be included. The computing system 700 also may include one or more communication systems 780 that allow the computing system 700 to communicate with other computing systems 790, for example, as the driving data monitor 110 aboard the vehicle 500 (FIG. 5) communicates with the remote computing system 650 and vice versa. As previously mentioned, the communication system 780 may include systems for wired or wireless communications. Available forms of communication media typically carry computer-readable instructions, data structures, program modules or other data in a modulated data signal such as a carrier wave or other transport mechanism and includes any information delivery media. The term “modulated data signal” may include a signal that has one or more of its characteristics set or changed in such a manner as to encode information in the signal. By way of illustrative example only and not of limitation, communication media may include wired media such as a wired network or direct-wired connection, and wireless media such as acoustic, radio frequency (RF), infrared and other wireless media. The term computer-readable media as used herein includes both storage media and communication media.

In further reference to FIG. 7, the computing system 700 may include global positioning system (“GPS”) circuitry 785 that can automatically discern its location based on relative positions to multiple GPS satellites. As described further below, GPS circuitry 785 may be used to determine a location of the vehicle 300 (FIG. 3) and to potentially identify alternative routes.

Referring to FIG. 8, the vehicle 500 (FIG. 5) may include an operator assist system 340 that includes a full automated driving system 850 to automatically drive the vehicle 500 and/or any number of operator assist features to aid an operator in driving the vehicle 500. The availability and use of these operator assist features by the operator may be considered by the driving analysis system 120 in generating the driving insurance offer 125 (FIGS. 1 and 4). The operator assist system 340 may include a forward collision warning system 802 to alert an operator, proceeding at a normal travel speed, of a stopped vehicle or other object in the road. The engagement of the forward collision warning system 802, or repeated use of the engagement of the forward collision warning system 802, may be indicative of operator inattention or of a route that may include dangers in the forms of abrupt vehicle stoppages. Similarly, the operator assist system 340 may include an automatic emergency braking system 804. While the forward collision warning system 802 alerts the operator to apply the brakes to avoid a stoppage or other object in the road, the automatic emergency braking system 804 actually automatically engages the brakes to stop the vehicle 500 of its own accord when a stoppage or other object is detected in the road. The engagement of the automatic emergency braking system 804 also may be indicative of operator inattention or of a route that may include dangers in the forms of abrupt vehicle stoppages.

The operator assist system 340 also may include an adaptive cruise control system 806. The adaptive cruise control system 806 automatically adjusts a cruising speed, set by the operator or the cruise control system, to reflect the speed of traffic ahead. For example, if an operator sets the adaptive cruise control system 806 to a posted highway speed of 65 miles per hour but, because of traffic, the speed of vehicles in the road ahead travel varies between 55 and 65 miles per hour, the adaptive cruise control system 806 will repeatedly adjust the cruising speed to maintain a desired distance between the vehicle and other vehicles in the road ahead. In various embodiments, the desired distance may be adjusted to reflect current conditions, including traffic and weather conditions.

The operator assist system 340 may include a lane departure warning system 808 that alerts an operator when the vehicle is veering close to or across a lane marker, presenting a potential hazard. Activation of the operator assist system 340 as logged in the trip data 115 for a route may indicate operator inattention. The operator assist system 340 may include a lane keeping assist system 810 that steers the vehicle to prevent the vehicle from veering close to or across a lane marker.

The operator assist system 340 may include a blind spot detection system 812 that alerts an operator of vehicles traveling in blind spots off the rear quarters of the vehicle to warn the operator not to change lanes in such cases. The operator assist system 340 may include a steering wheel engagement system 814 that detects when an operator has released the wheel. Release of the wheel may be logged to the trip data 115 as an indication of operator inattention. The operator assist system 340 also may include a traffic sign recognition system 816 that, for example, recognizes stop signs or speed limit signs. Availability of the traffic sign recognition system 816 may make travel on surface or neighborhood streets preferable because the system will help reduce the likelihood of an accident caused by missing a sign or traveling at an inappropriate speed.

The operator assist system 340 also may include a rear cross-traffic alert system 818 to apprise an operator of the approach of other vehicles when the vehicle is moving out of a space. Similarly, the operator assist system 340 may include a backup warning system 820 that warns the operator when the vehicle is approaching an object behind the vehicle. The operator assist system 340 may include an automatic high-beam control system 822 to de-activate and re-activate high beams as other cars approach and then pass by. Availability of such a system may reduce the likelihood of incidents during travel on highways or surface streets with insufficient or no lighting. The operator assist system 340 may also include a manual park assist system 824 to aid an operator in parking the vehicle.

Referring to FIG. 9, and as previously described, the driving data monitor 110 may receive data from a number of sensors 900 to monitor driving behavior. As previously described, some of the sensors 900 may include vehicle sensors 111 mounted on the vehicle 500 (FIG. 5) and/or coupled with its systems, such as the drive system 530. Others of the sensors, such as accelerometers or other sensors, may be housed within the driving data monitor 110 regardless of whether the driving data monitor 110 is incorporated into the vehicle 500 or is a separate computing device, such as a portable computer, tablet computer, smartphone, or smartwatch, that may be transported aboard the vehicle 500.

In addition to a GPS sensor 930 that may be used to track position or movement, the sensors 900 may include an accelerometer 932 to detect rapid accelerator or deceleration that may indicate overly-aggressive driving, hard braking, abrupt cornering, or other actions that may indicate aggressiveness or inattention of the operator or dangerous traffic patterns. The sensors 900 may include a gyroscope 934 to detect abrupt changes of direction indicative of a treacherous road, sharp lane changes, or abrupt turns.

The sensors 900 also may include at least one following distance sensor 936 that determine how closely the vehicle 500 follows other vehicles. The following distance sensor 936 may use any technology that can determine following distance from another vehicle, such as radar, LIDAR, optical measurement made using cameras or other optical sensors, ultrasonic measurement, laser measurement, or any other technology that can be used to determine following distance from another vehicle. Logging the following distance data for particular routes may indicate whether the traffic conditions on those roads suggest an increased risk of loss, whether the operator tends to follow other vehicles too closely on certain routes, or other issues.

The sensors 900 also may include device sensors, such as tire pressure sensors 938 to monitor whether the tires are inflated to a recommended level. The sensors 900 may include miscellaneous device sensors 940 to determine whether other systems, such as the lights, horn, and wipers have been used on particular routes. The sensors 900 may also include a seatbelt sensor 942 to indicate whether the occupants wore seatbelts on a particular trip. The sensors 900 may also include a phone usage sensor 944 (which may take the form of an app executing on the phone) to report whether the operator was handling or operating the operator's phone while driving. The sensors 900 may include an airbag deployment sensor 946 or a collision sensor 948 to report a catastrophic event that resulted in a collision and/or a serious collision that warranted deployment of the airbag.

Finally, the sensors 900 may include one or more operator condition sensors 950. The operator condition sensor 950 may communicate with a health monitor device, which may be included in a smartwatch or be incorporated in a wearable device, such as a health monitor band. These devices may monitor heart rate or other operator conditions. The operation condition sensor 950 also may include a blood alcohol sensor or similar devices to gauge whether an operator may be impaired. In sum, the driving analysis system 120 (FIGS. 1 and 4) may evaluate information provided by the sensors 900 to evaluate the choices and the driving behavior of the operator.

Additionally, in various embodiments, the sensors 900 may include an offroad sensor 952. An extent to which a vehicle is operated offroad and the manner in which the vehicle is operated in these conditions may provide further indicial of the driving behavior of the operator.

Table (1) presents a list of data that may be presented by the operator assist system 340 and/or the vehicle data system 380 as described with reference to FIGS. 6 and 7. Table (1) includes a data field that may be logged and, for example, a frequency with which the data is sampled and/or stored.

TABLE 1

| | | Minimum | |
|---|---|---|---|
| | | Reporting | |
| Field | Description | Frequency | |
| Driver ID | Unique identifier for each driver | NA | |
| | when available | | |
| Trip ID | Unique identifier for a specific trip | NA | |
| Trip Start | Start date and time of trip | NA | |
| Trip End | End date and time of trip | NA | |
| Road Speed | 1 Hz using multiple sensors | 1 | Hz |
| GPS Accuracy | | 1 | Hz |
| GPS Speed | | 1 | Hz |
| GPS Altitude | | 1 | Hz |
| GPS Heading | | 1 | Hz |
| GPS Latitude | | 1 | Hz |
| GPS Longitude | | 1 | Hz |
| Accelerometer | | 10 | Hz |
| Gyroscope | | 10 | Hz |
| Collision/Impact | Calculate in real-time based on | | |
| Sensors | available sensor and contextual | | |
| | data | | |
| Rear-ended | Calculate in real-time based on | | |
| | available sensor and contextual | | |
| | data | | |
| Side impact | Calculate in real-time based on | | |
| | available sensor and contextual | | |
| | data | | |
| Airbag Sensors | | 10 | Hz |
| Vehicle Roll- | Calculate in real-time based on | | |
| over | available sensor and contextual | | |
| | data | | |
| Vehicle Spin-out | Calculate in real-time based on | | |
| | available sensor and contextual | | |
| | data | | |
| Vehicle Security | Upon alarm triggering | 1 | Hz |
| Breach | | | |
| Odometer | Trip start/end | NA | |
| Impact Sensor | As it happens | 10 | Hz |
| Event | | | |
| Driver Seatbelt | On on/off | 1 | Hz |
| Event | | | |
| Passenger | On on/off | 1 | Hz |
| Seatbelt | | | |
| Event | | | |
| Following | Identify driving behavior to | 10 | Hz |
| Distance | segment risk factor based on | | |
| | following distance, relative to | | |
| | speed | | |
| Hard Braking | Calculate hard brake events | 10 | Hz |
| Rapid | Calculate rapid acceleration events | 10 | Hz |
| Acceleration | | | |
| Aggressive | Calculate aggressive cornering | 10 | Hz |
| Cornering | | | |
| Speed above | Identify time above Posted Speed | Post | |
| PSL | Limit | processing | |
| Excessive Speed | Identify time above a fixed speed | 1 | Hz |
| | limit | | |
| Steering Wheel | | 1 | Hz |
| Engagement | | | |
| System | | | |
| Forward | | 10 | Hz |
| Collision | | | |
| Warning | | | |
| Lane Departure | | 10 | Hz |
| Warning | | | |
| Rear Cross | | 10 | Hz |
| Traffic on/Off | | | |
| Rear Cross | Identify when rear cross traffic | 10 | Hz |
| Traffic Warning | event occurs | | |
| Traffic Sign | | 1 | Hz |
| Recognition | | | |
| System | | | |
| Manual Park | | 10 | Hz |
| Assist On/Off | | | |
| Manual Park | Identify when manual park warning | 10 | Hz |
| Assist Warning | event occurs | | |
| Navigation in- | | 1 | Hz |
| use | | | |
| Auto | | 10 | Hz |
| Emergency | | | |
| Braking | | | |
| Engaged | | | |
| Low Tire Air | Tire pressure below certain | 1 | Hz |
| Pressure | threshold (Front right, Front left, | | |
| | Rear right, Rear left) | | |
| Autonomous | On on/off | 10 | Hz |
| Driving Mode | | | |
| On/Off | | | |
| Adaptive Cruise | On on/off | 10 | Hz |
| Control | | | |
| Blindspot | On on/off | 10 | Hz |
| Monitoring | | | |
| On/Off | | | |
| Blindspot | Identify when blindspot event | 10 | Hz |
| Warning | occurs | | |
| Backup Warning | | 1 | Hz |
| System | | | |
| Headlights | On on/off | 10 | Hz |
| On/Off | | | |
| Fog Lights | On on/off | 10 | Hz |
| On/Off | | | |
| Automatic High | | 1 | Hz |
| Beam Control | | | |
| System | | | |
| Rain Sensor | | 10 | Hz |
| Windshield | On on/off | 10 | Hz |
| Wipers | | | |
| On/Off | | | |

Some or all of the information listed in Table (1), along with other information, may be used to evaluate the trip data 115 (FIGS. 1-4) to determine a score for the operator(s). The trip data 115 thus may be scored.

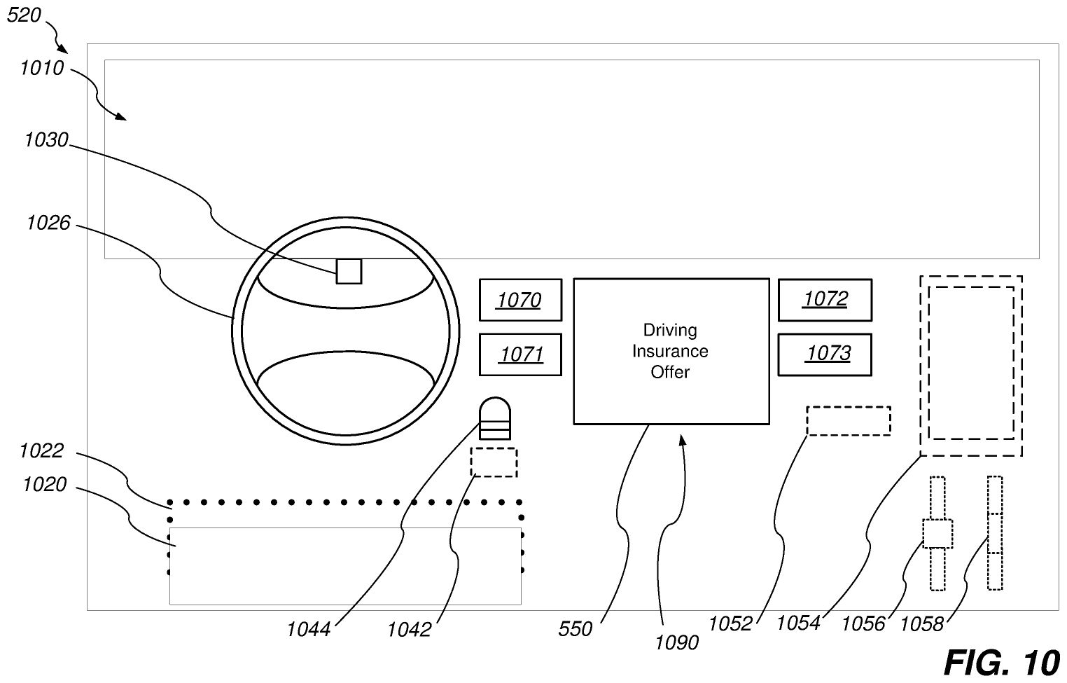

Referring to FIG. 10, in various embodiments, the cabin 520 of the vehicle (FIG. 5) includes an operator display and input system 550 (FIG. 5) and a number of devices for identifying the operator and performing other functions. The cabin 520 familiarly includes a windshield 1010 and an operator's seat 1020, as well as a steering wheel 1026 and other controls, such as the accelerator, brake pedal, and switches to operate the headlights, wipers, etc. (not shown).

To identify the operator, the cabin 520 may include an operator identification system 570 (FIG. 3) that includes some or all of a number of identification devices. One or more cameras or other imaging device 1030 may be positioned to image the operator, who may be identified using image recognition. The operator also may be identified by the operator adjusting the operator's seat 1020 to an adjusted position 1022 that is favored by a particular operator. The position may be settable by selecting one of a number of memory buttons (not shown) assignable to each of a number of operators. Also, the passenger cabin 320 may include a key fob identifier 1042 that not only recognizes that a key fob 1044 is authorized to operate the vehicle, but to recognize when the key fob 1044 is that assigned to a particular operator. The key fob 1044 may, for example, include an individualized radio frequency identification (RFID) tag and the key fob identifier 1042 may include an RFID reader. Also, the cabin 520 may include a device connection system 1052 that, in addition to, for example, enabling a smartphone 1054 to interact with the vehicle's entertainment system or other systems, identifies whether the smartphone 1054 is associated with a particular operator of the vehicle. The device connection system 1052 also may be used to identify a presence of a smartwatch 1056 or a health monitor device 1058, such as a health monitor band or other wearable device, associated with a particular operator to identify the operator.

The operator display and input system 550 provides a range of information about routes, vehicle operations, and, in various embodiments, the driving insurance offer 125 (FIGS. 1 and 4) as previously described. In various embodiments, the operator display and input system 550 may be a touchscreen that enables the operator to choose control various vehicle operations. In various embodiments, the cabin 520 may include other controls, such as 1070-1073 to engage or disengage automated driving or operator assist features or perform other functions. The controls 1070-1073 may be on a dashboard 1090, on the steering wheel 1026, or on another surface in the cabin 520. It will be appreciated that the controls 1070-1073 may be only a subset of the controls available or needed to engage automated driving or the operator assist features, or some or all of these features may be controllable via the operator display and input system 550.

In various embodiments, various devices may be used to identify distracted driving on the part of the operator. For example, the one or more cameras or imaging devices 1030 may be used to determine if the operator is not observing the road, is eating while driving, applying cosmetics while driving, or is distracted by a phone or other objects. Similarly, the device connection system 1052 may communicate with a smartphone 1054, smartwatch 1056, or health monitor device 1058 to determine if the operator is manipulating one of these devices while driving. The smartwatch 1056 or health monitor device 1058 may communicate via the device connection system whether the operator is moving excessively, possibly indicating the operator is, for example, gesturing excessively as part of a conversation or moving along with music, in a way that may be indicative of distracted driving.

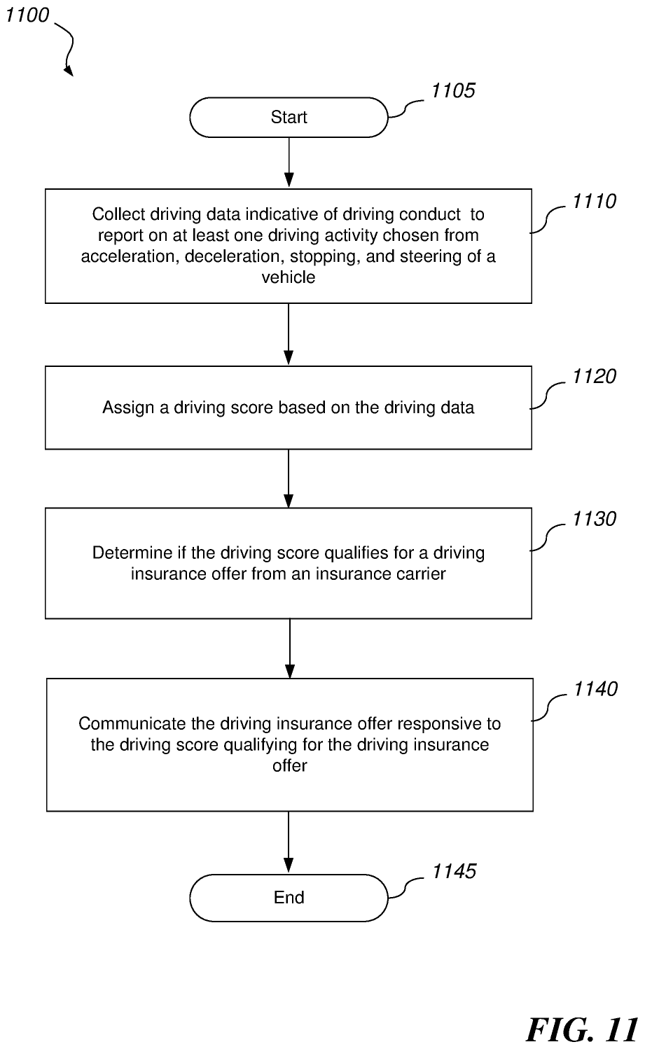

Referring to FIG. 11, an illustrative method 1100 of collecting and evaluating driving data for the possible generation of a driving insurance offer 125 is presented. The method 1100 starts at a block 1105. At a block 1110, driving data is collected that is indicative of driving conduct to report on at least one driving activity chosen from acceleration, deceleration, stopping, and steering of a vehicle. At a block 1120, a driving score is assigned based on the driving data. At a block 1130, it is determined if the driving score qualifies for a driving insurance offer 125 from an insurance provider. At a block 1140, the driving insurance offer 125 is communicated responsive to the driving score qualifying for the driving insurance offer 125. The method 1100 ends at a block 1145.

It will be appreciated that the detailed description set forth above is merely illustrative in nature and variations that do not depart from the gist and/or spirit of the claimed subject matter are intended to be within the scope of the claims. Such variations are not to be regarded as a departure from the spirit and scope of the claimed subject matter.

What is claimed is:

1. A system comprising:
a driving data monitor configured to receive driving data on driving conduct for at least one operator of a vehicle; and
a driving analysis system configured to:
receive the driving data from the driving data monitor:
assign a driving score based on the driving data;
determine if the driving score qualifies for a driving insurance offer from an insurance provider; and
communicate the driving insurance offer via an interface responsive to the score qualifying for the driving insurance offer.

2. The system of claim 1, wherein the driving insurance offer includes a driving insurance offer chosen from one of a driving insurance offer for a particular operator and a plurality of operators of a particular vehicle.

3. The system of claim 1, the driving analysis system is configured to assign the driving score based at least in part on detecting:
driving speed compared to a posted speed limit and a speed limit adjusted for conditions;
acceleration relative to a predetermined acceleration limit;
braking relative to a predetermined deceleration limit; and
engaging in distracted driving.

4. The system of claim 1, wherein the driving data monitor includes at least one of:
an onboard system integrated into operations of a vehicle; and
a computing device transportable aboard the vehicle including one of a portable computer, a tablet computer, a smartphone, and a smartwatch.

5. The system of claim 1, wherein the driving data monitor received the driving data from a plurality of sensors chosen from at least one of:
vehicle sensors incorporated into the vehicle; and
monitor sensors incorporated into the driving data monitor.

6. The system of claim 1, wherein the driving data monitor is further configured to identify a particular operator using an identifier chosen from at least one of:
a key fob identifier configured to identify a key fob assigned to the particular operator;
a communications identifier configured to detect a presence aboard the vehicle of one of a smartphone, a smartwatch, and a health monitor device associated with the particular operator;
a seat position identifier detector configured to identify the particular operator based on a position of a driver's seat associated with the particular operator; and
an image of the particular operator determinable by an imaging system.

7. The system of claim 1, wherein the driving analysis system is further configured to determine if:
the operator has received a previous driving insurance offer from the insurance provider before determining if the driving score qualifies the operator for the driving insurance offer; and
the driving insurance offer includes a lower cost than the previous driving insurance offer before communicating the driving insurance offer to the operator.

8. The system of claim 1, wherein the driving analysis system is further configured to incorporate in the driving score an extent to which at least one of:
operator assist capabilities were engaged;
corrective action was taken by the operator assist capabilities automated driving was engaged; and
distracted driving was detected.

9. A vehicle comprising:
a cabin configured to receive at least one occupant;
a drive system configured to motivate, accelerate, decelerate, stop, and steer the vehicle;
a vehicle control system configured to allow an operator to direct operations of the vehicle;
a driving data monitor configured to receive driving data on driving conduct; and
a driving analysis system configured to:
receive the driving data from the driving data monitor:
assign a driving score based on the driving data;
determine if the driving score qualifies for a driving insurance offer from an insurance provider; and
communicate the driving insurance offer via an interface responsive to the score qualifying for the driving insurance offer.

10. The vehicle of claim 9, wherein the driving insurance offer includes a driving insurance offer chosen for one of a particular operator and operators of a particular vehicle.

11. The vehicle of claim 9, the driving analysis system is configured to assign the driving score based at least in part on detecting:
driving speed compared to a posted speed limit and a speed limit adjusted for conditions;
acceleration relative to a predetermined acceleration limit;
braking relative to a predetermined deceleration limit; and
engaging in distracted driving.

12. The vehicle of claim 9, wherein the driving data monitor includes at least one of:
an onboard system integrated into operations of a vehicle; and
a computing device transportable aboard the vehicle including one of a portable computer, a tablet computer, a smartphone, and a smartwatch.

13. The vehicle of claim 9, wherein the driving data monitor is further configured to identify a particular operator from at least one identifier chosen from:
a key fob identifier configured to identify a key fob assigned to the particular operator;
a communications identifier configured to detect a presence aboard the vehicle of one of a smartphone, a smartwatch, and a health monitor device associated with the particular operator;
a seat position identifier detector configured to identify the particular operator based on a position of a driver's seat associated with the particular operator; and
an image of the particular operator determinable by an imaging system.

14. The vehicle of claim 9, wherein the driving data processing system is further configured to determine if:
a previous driving insurance offer was received from the insurance provider before determining if the driving score qualifies for the driving insurance offer; and
the driving insurance offer includes a lower cost than the previous driving insurance offer before communicating the driving insurance offer.

15. The vehicle of claim 9, wherein the driving analysis system is further configured to incorporate in the driving score an extent to which at least one of:
operator assist capabilities were engaged;
corrective action was taken by the operator assist capabilities
automated driving was engaged; and
distracted driving was detected.

16. The vehicle of claim 9, wherein the driving analysis system is further configured to incorporate in the driving score an action taken by the operator assist system.

17. A computer-implemented method comprising:
collecting driving data indicative of driving conduct to report data on at least one driving activity chosen from acceleration, deceleration, stopping, and steering of a vehicle;
assigning a driving score based on the driving data;
determining if the driving score qualifies for a driving insurance offer from an insurance provider; and
communicating the driving insurance offer responsive to the driving score qualifying the operator for the driving insurance offer.

18. The computer-implemented method of claim 17, further comprising:
extending the driving insurance offer to a particular operator; and
extending the driving insurance offer to a plurality of operators of a particular vehicle.

19. The computer-implemented method of claim 17, further comprising assigning the driving score based at least in part on evaluating the operator based one at least one of:
driving speed compared to a posted speed limit and a speed limit adjusted for conditions;
acceleration relative to a predetermined acceleration limit;
braking relative to a predetermined deceleration limit; and
engaging in distracted driving.

20. The computer-implemented method of claim 17, further comprising determining whether:
a previous driving insurance offer has been received from the insurance provider before determining if the driving score qualifies for the driving insurance offer; and
the driving insurance offer includes a lower cost than the previous driving insurance offer before communicating the driving insurance offer.
